(19) United States
(12) Patent Application Publication
(10) Pub. No.: US 2007/0275098 A1
(43) Pub. Date: Nov. 29, 2007

(54) Formulation and methodology for the treatment for eye impairment symptoms

(75) Inventor: Brian Banks, Dillon, CO (US)

Correspondence Address: John D. Long, PATENT COUNSEL;LONG & CHYBIK, Suite 115, 1575 Delucchi Lane, Reno, NV, 89502, US

(73) Assignee: T.R.P. COMPANY, INC, a Nevada Corporation, Reno, NV (US)

(21) Appl. No.: 11/805,123

(22) Filed: May 21, 2007

Related U.S. Application Data
(60) Provisional application No. 60/802,239, filed on May 19, 2006.

(51) Int. Cl. A61K 36/75; A61K 33/14

(52) U.S. Cl. 424/675; 424/680; 424/725

(57) ABSTRACT

One possible embodiment of the invention could comprise a homeopathic composition suitable for the treatment of the symptoms of eye impairment conditions which comprises Cineraria Maritima in five times potency; Calcium Fluoride in thirteen times potency; Conium Maculatum in six times potency; Euphrasia Stricta in six times potency; Gelsemium Sempervirens in six times potency; Sodium Chloride in six times potency; and Ruta Gravelens in six times potency. This embodiment could possibly include a methodology of applying a homeopathic effective amount of the above homeopathic composition to eye.

### CROSS-REFERENCES TO RELATED APPLICATIONS

This present application claims the benefit under 35 U.S.C. § 119(e) of U.S. Provisional Patent Application No. 60/802239, filed on May 19, 2006, contents of which are relied upon and incorporated by reference.

### STATEMENT REGARDING FEDERALLY SPONSORED RESEARCH OR DEVELOPMENT

Not Applicable

### REFERENCE TO A “MICROFICHE APPENDIX”

Not Applicable.

1. Field of the Invention

The present invention generally relates to formulation and methodology for eye treatment and more specifically, it may relate to the treatment of symptoms of eye impairment.

2. Background

As a person ages, the person may become aware that their eyes may have developed symptoms of eye impairment. The person may notice that their eyes appear to be easily irritated (as seen from redness, itchiness, or soreness of the eye); appear to suffer bouts of dryness; and seem to experience a reduction in visual capability both in day and night vision. While soreness, dryness and other irritation of the eye may be temporarily treated by conventional remedies, long-term conventional medical treatment of such symptoms, especially in regards to the reduction in visually capability, may not be so easily obtained.

One possible causation theory for reduction in visual clarity or perception may be the impairment of accommodation, the eye's general ability to change its focus to see correctly objects at different distances. In accommodation, the eye may use its ciliary muscles to automatically bend or straighten the eye or crystalline lens (which is somewhat elastic and has curved front and back sides) to bend the light passing through the lens to properly focus that light on the back of the eye known as the retina. However, when a person reaches middle age, the person's ciliary muscles may lose some of its power to shape eye or crystalline lens (e.g., change the curvature of the front and back of the lens) as well as the person's eye lens may lose some its elasticity as well. It is also thought that this loss of eye lens elasticity may be due to a decrease of oxygen and nutrient flow via the aqueous fluid to the lens. When this impairment of the focusing of the lens (or of the eye accommodation process) occurs, the resulting condition is known as presbyopia.

Conventional medicine may offer several mechanical and surgical solutions for this condition. Some presbyopia sufferers may find it necessary to hold reading materials further away, or require larger print and more light to read by. People who do not need glasses for distance vision may only need half glasses or reading glasses. For those with other visions issue (e.g., nearsightedness or myopia) one possible mechanical solution may be corrective eyewear, which may have multifocal lenses (such as bifocal or trifocal lenses) or progressive lenses. Another mechanical remedy is monovision corrected eyewear lens wherein one eye has vision corrected for near vision while the other eye is corrected for far vision.

Surgical remedies for presbyopia may include monovision laser eye surgery, which surgically corrects one eye for near vision and the other eye for distance vision. Other surgical remedies may include the placement of intraocular lens implant in both eyes.

In addition to the above conventional medical remedies, there is a continued consumer interest in the use and development of complementary medicines that are generally underutilized by the conventional medicine practitioners. Typically, complementary medicine may be used to supplement the primary or conventional medical treatments. One such field of complementary medicine is the homeopathy, which may use medicine remedies or formulations that have ingredients or elements that are generally not used in conventional medicine. Further, such homeopathic formulations may present their active ingredients in concentrations much lower than the concentrations substantially used in conventional medicine pharmaceuticals.

Homeopathy was substantially first developed 1790 where it was noticed that a prevalent treatment for malaria at that time was ingestion of bark of the cinchona tree (much later, quinine, a major conventional medicine anti malarial drug, was substantially extracted and refined into higher concentrations from this bark). At that time, conventional medicine held that the bark's effectiveness was due to its very bitter taste. It was then generally noticed other medicines were equally bitter, but they were not particularly useful in the treatment of malaria, and that other aspects of this remedy must be responsible for its medicinal properties. It was discovered that a person not having the corresponding disease (e.g., malaria) took (e.g., ingested) of the remedy (e.g., the cinchona bark) that person then substantially exhibited all the disease's (e.g., malaria) symptoms (e.g., chills, fever, palpitations, sweats, etc). It was further rationalized that it could be possible to develop a remedy for an illness based on utilizing those elements, which substantially mimicked the symptoms of the disease when taken by a healthy person. The remedy, however, could contain dosages of the selected element(s) in quantity(ies) much lower than those levels that caused the symptoms in otherwise healthy individuals taking the selected elements.

Practitioners of Homeopathy (e.g., Homeopaths) may use the above homeopathic remedy development doctrine to individually identify various potential homeopathic elements for use in a remedy. These provings (e.g. testing and recording of the results for various potential homeopathic ingredients or elements) were then collected and organized in various texts. One such text, the Homeopathic Pharmacopoeia of the United States (“HPUS”), is recognized by the United States Government in that it is used by the US Food and Drug Administration (FDA) as its official homeopathic remedy guideline for the classification and regulation of the manufacture homeopathic medicines. Today's Homeopath will use the HPUS to select substantially one or more approved ingredients at regulated levels as a basis to form generally a homeopathic remedy.

Many times, the source of the ingredients for a homeopathic remedy could be a naturally occurring organic substance such as an herb or the like. In such cases, the succus (e.g., an extract) could be produced from homeopathic element source such as the homeopathic recognized herb or the like by taking a sample of the said substance and squeezing or otherwise removing the juice or the extract from the sample. In some cases, the succus could be further processed or titrated [e.g., preparing the smallest amount of a homeopathic element of known concentration that is required to bring about a given effect (e.g., replicate illness symptoms) in reaction in a patient] to form a tincture.

The tincture of the homeopathic element could then be further diluted by factors of 10. For example, a 10² dilution factor of the tincture could be made by generally having one part of the tincture placed into 100 parts of a diluent (e.g., purified water) (e.g., 1/10² dilution). The resulting diluted volume could then go through a “potentizing” wherein then diluted volume could be place and sealed into a heavy glass jar for further mixing. The homeopathic remedy preparer could then lift the sealed jar up above his or her head and then slap the jar down onto a padded surface (e.g., folded-over leather pad) several times. This potentizing may then induce an agitation or turbulence in the diluted volume, which is believed to mix properly a diluted solution into the dilution volume. Once the potentizing is completed for that particular dilution, the N times or N× dilution could then be referenced as a N times potency (wherein N is generally a whole number.)

The dilution process could be repeated again (for another 1× dilution) and be followed by a respective potentizing action to achieve the HPUS prescribed ultimate dilution for the selected element or ingredient. Once each of the various active ingredients or elements are made to their respective proper dilutions, they could be placed in an appropriate delivery matrix (e.g., a saline solution containing appropriate preservative, gel base for a salve, etc) to form the homeopathic remedy to be given to the patient. The selection of the matrix and the amount of appropriately diluted homeopathic ingredients to place within the matrix could be decided by persons having ordinary skill in the art.

In using the homeopathic remedy analysis, the homeopath generally has to decide on what observable indicators present in the sick patient correlates the respective symptoms of an imbalance of the body or disease. The homeopath, as a diagnostician, generally examines a wider field of symptoms (e.g. the emotional state of the patient and the like) than does the conventional medical practitioner. Once the present indicators of the patent are properly identified as illness symptoms, the homeopath then tries to match those identified symptoms to those symptoms caused by homeopathic ingredients as documented in the HPUS. In selecting the proper active ingredients or elements for the homeopathic remedy, homeopath has to take into account that some homeopathic ingredients may mimic one or more symptoms of illness.

What is needed therefore is a homeopathic remedy that can be used to aid the relief of one or more symptoms accompanying the impairment of a person's eyes that may occur as the person ages.

### SUMMARY OF ONE EMBODIMENT OF THE INVENTION

### Advantages of One or More Embodiments of the Present Invention

The various embodiments of the present invention may, but do not necessarily, achieve one or more of the following advantages:

the ability to possibly increase nutrient and oxygen flow to the crystalline lens;

the provide a remedy which may possibly reduce eye irritation;

the ability to possibly improve an impaired night vision;

provide a remedy which could possibly help reduce an impairment of the accommodation of the eye;

the ability to possibly reduce redness of the eye;

provide a remedy that may be used to treat the symptoms of presbyopia.

These and other advantages may be realized by reference to the remaining portions of the specification, claims, and abstract.

### BRIEF DESCRIPTION OF DRAWINGS

Without restricting the full scope of this invention, the preferred form of this invention is illustrated in the following drawings:

FIG. 1 substantially shows a list of the active homeopathic ingredients or elements for the composition that could comprise the invention.

### BRIEF DESCRIPTION OF ONE EMBODIMENT OF THE PRESENT INVENTION

In one aspect, the invention could be an eye distress relief homeopathic composition, which could be used to possibly treat symptoms of eye impairment (e.g., dry eye, poor night vision, red eye, eye irritation, blurry vision caused by presbyopia, and the like) substantially comprising of at least the diluted tinctures of several homeopathic ingredients, namely (1) Cineraria Maritima (e.g., Dusty Miller), (2) Calcium Floride (e.g., CaF₂), (3) Conium Maculatum (e.g., Poison Hemlock), (4) Euphrasia Stricta (e.g., Eyebright) (5) Gelsemium sempervirens (e.g., Yellow Jasmine), (6) Sodium Chloride (e.g., NaCl), and (7) Ruta Gravelens (e.g., Garden Rue).

In another aspect, the invention could be a method of treating eye distress or impairment in a human comprising administrating an homeopathic effective amount of the aforesaid composition to a human.

The above description sets forth, rather broadly, a summary of one embodiment of the present invention so that the detailed description that follows may be better understood and contributions of the present invention to the art may be better appreciated. Some of the embodiments of the present invention may not include all of the features or characteristics listed in the above summary. There are, of course, additional features of the invention that will be described below and will form the subject matter of claims. In this respect, before explaining at least one preferred embodiment of the invention in detail, it is to be understood that the invention is not limited in its application to the details of the construction and to the arrangement of the components set forth in the following description. The invention is capable of other embodiments and of being practiced and carried out in various ways. Also, it is to be understood that the phraseology and terminology employed herein are for the purpose of description and should not be regarded as limiting.

### DESCRIPTION OF CERTAIN EMBODIMENTS OF THE PRESENT INVENTION

In the following detailed description of the preferred embodiments, specific embodiments may be set fourth in which the invention may be practiced. It is to be understood that other embodiments may be utilized and structural changes may be made without departing from the scope of the present invention.

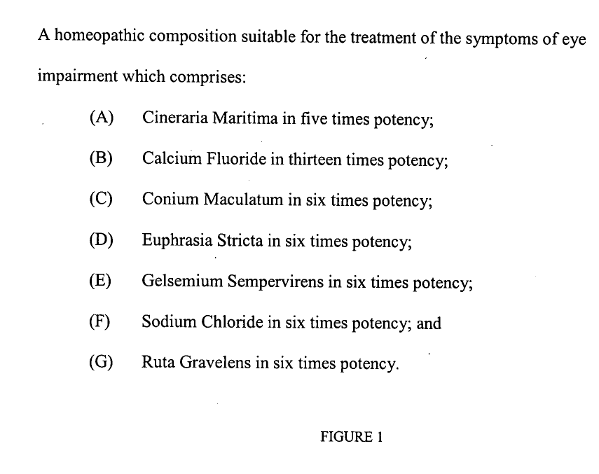

As substantially shown in FIG. 1, the present invention could comprise of a homeopathic composition of the following active ingredients (1) Cineraria Maritima (Dusty Miller), (2) Calcium Floride (CaF₂), (3) Conium Maculatum L. (Poison Hemlock), (4) Euphrasia Stricta (Eyebright) (5) Gelsemium Sempervirens (Yellow Jasmine), (6) Sodium Chloride (NaCl), and (7) Ruta Gravelens (Garden Rue), which may be available from Apothecara Natural, Inc. of Woodbine, Iowa, USA.

In one possible embodiment, the elements or active ingredients of the above composition could be diluted by standard homeopathic methods (e.g., wherein a 1× dilution could mean taking 1 part by weight of an active homeopathic ingredient and diluting into 10 parts by weight of HPUS approved diluent (e.g., filtered water) to substantially create a 10% by weight dilution of the initial homeopathic amount. Similarly, a 2× dilution could be taking 1 part by weight of 1× dilution and generally placing it into 10 parts of diluent to substantially create a 3× or 1% by weight dilution. A 1 part by weight of the 3× dilution could generally be placed in a 10 parts by weight diluent to substantially create 0.1% by weight dilution.

In such a manner, the N× (e.g., N times) or N times homeopathic dilution (wherein N is generally a whole number) indicates that the dilution procedure had occurred N times for that particular element. Each dilution could also be followed by a respective potentizing. A dilution followed by a respective potentizing could be also known as potency [e.g., 1× dilution or 1 times dilution with following respective potency (as discussed above) could be also be known as a 1× potency or one times potency; a 5× dilution or a five times dilution followed by respective potency could be also known as a 5× potency or a five times potency; and the like]. Each of the dilutions could vary with a tolerance of ±10%.

At least one embodiment of the invention could have the homeopathic composition delivered to surface of the subject's eye via a liquid format (e.g., drop or stream). In one possible embodiment, the liquid format could have a saline solution delivery matrix that contains an amount of the homeopathic composition. Those having ordinary knowledge in the art could determine the type of saline solution delivery matrix and the amount of homeopathic composition that it may contain to form the liquid format.

One possible saline solution delivery matrix could be comprised of purified water and sodium chloride, the matrix further containing other non-active ingredients such as pH buffers, preservatives and the like as known by those skilled in the art for use in eye drop medication, remedies, and the like. Another possible composition of a saline solution delivery matrix could comprise of a purified water base further containing the non-active ingredients sodium chloride, benzalkonium chloride (0.01%), boric acid, edentate disodium, and sodium citrate. In such matrix examples, hydrochloric acid and/or sodium hydroxide could be used to adjust the overall pH of the liquid format.

If the user is wearing contact lens while using the invention, another possible example of such saline solution delivery matrix could be a purified water base with sodium chloride, further containing other non-active ingredients such as hypromellose and glycerin, with sorbic acid (0.25%) and edentate disodium (0.1%) as the preservatives.

One possible embodiment of the invention could be a composition comprising of a liquid mixture of the above-named seven (7) active homeopathic ingredients [i.e., (1) Cineraria Maritima (e.g., Dusty Miller), (2) Calcium Fluoride (e.g., CaF₂), (3) Conium Maculatum L.(e.g., Poison Hemlock), (4) Euphrasia Stricta (e.g., Eyebright) (5) Gelsemium Sempervirens (e.g., Yellow Jasmine), (6) Sodium Chloride (e.g., NaCl), and (7) Ruta Gravelens (e.g., Garden Rue)]. In at least one embodiment of the invention, the composition may further contained within a saline solution matrix as described above to form a liquid format for delivery to the subject's eye.

The herb Cineraria Maritima (common name Dusty Miller) could be prepared by having its tincture, as denoted HPUS, made from a succus of the fresh plant that could then be diluted by factor of 5× (e.g., reduced 1 part tincture per 100,000 parts of purified water diluent or a 1/10⁵ dilution). With appropriate accompanying potency, Cineraria Maritima could be at five times (5×) potency for the composition.

One possibly approved HPUS succus for Cineraria Maritima could be making a mixture containing by weight extracted juice from fresh whole plant excluding roots (87 per cent), strong alcohol (8 per cent), glycerine (5 per cent), and phenyl mercuric nitrate (0.001 per cent). Using an HPUS approved succus, the tincture could be prepared combining the following: 100 grams of the Cineraria Maritima, 500 ml of purified water, and 635 ml of strong alcohol to make substantially a base or Mother Tincture.

The herb Conium Maculatum (e.g., commonly known as poison hemlock) could have as its tincture as denoted HPUS could be formed from a succus made from a fresh whole plant. The Conium Maculatum for the homeopathic composition could be at six times (6×) potency.

One possible succus for Conium Macalatum could be 100 ml extracted from 100 grams of solids of Conium Maculatum (e.g. fresh whole plant). One possible Mother Tincture for Conium Macalatum could be a solution of 100 grams of the Conium Maculatum in coarse powder, 400 ml of purified Water, and 637 ml of string alcohol.

The herb Euprasia Officinalis (common name Eyebright) could be prepared by have a tincture of a succus of a fresh plant, as HPUS denoted, diluted by a factor of 6×, or 1 part tincture to 1,000,000 parts of purified water diluent (e.g., 1/10⁶). With accompanying potency for the respective dilution, the Euprasia Officinalis could be six times (6×) potency for the homeopathic composition of the invention.

One possible succus for Euprasia Officinalis could be the 200 ml moisture content of fresh plant from which is extracted 100 grams of solids. One possible tincture for Euprasia Officinalis could be a solution (e.g., Mother Tincture) generally containing 100 grams of the Euprasia Officinalis in course powder, 400 ml of purified water; and 635 ml strong alcohol.

The herb Ruta Gravelens (common name Bitter herb) could be a tincture from a whole fresh plant diluted by a 6× factor or 1 part tincture per 1,000,000 parts of purified water diluent (e.g., 1/10⁶) With proper potency accompanying the respective dilution, the homeopathic element Ruta Gravelens for this composition could be. six times (6×) potency.

One possible tincture for Ruta Gravelens could be a base or Mother Tincture comprising of 100 grams of the Ruta Gravelens in coarse powder, 300 ml of purified water, and 730 ml of strong alcohol 100 grams.

The herb Gelsemium Sempervirens (common name Bignonia) could be a tincture of the bark of the root of the herb Gelsemium Sempervirens diluted 6× [e.g., one part tincture per 1,000,000 parts of purified water diluent (e.g., 1/10⁶).] With corresponding proper potency being imparted to the respective dilution, the homeopathic active element Gelsemium Sempervirens for this composition could be at six times (6×) potency.

One possible tincture for Gelsemium Sempervirens could be a base or a Mother Tincture generally comprising of 10 g of powder form of the Gelsemium Sempervirens plus 400 ml of purified water and 635 ml of strong alcohol.

The chemical compound Calcium Fluoride (CaF₂) (e.g., also known as Floride of Lime or Calcarea Fluorica) has its tincture, as denoted HPUS, as being diluted by a factor of 13×, or 1 part per 10,000,000,000,000 parts purified water diluent (e.g., 1/10¹³). With proper potency accompanying the respective dilutions, Calcium Fluoride in the homeopathic composition could be at thirteen times (13×) potency.

A thousand gram tincture of Calcium Fluoride could comprise 100 grams of Calcium Fluoride in coarse powder form and 900 grams of lactose (Cl₂H₂₂O₁₁).

The chemical compound Sodium Chloride (NaCl) (e.g., common salt) may have its tincture, as HPUS denoted, diluted by a factor of 6× or 1 part tincture to 1,000,000 parts of purified water diluent (e.g., 1/10⁶). With corresponding proper potency being imparted to the respective dilution, Sodium Chloride in the homeopathic composition could be six times (6×) potency.

A possible tincture for this active homeopathic ingredient could comprise of 100 grams of Sodium Chloride in crystal form combined with 900 grams of lactose (C₁₂H₂₂O₁₁).

As discussed-above, for each 1× dilution of every active homeopathic ingredient, the resulting diluted volume could be potentized.

Once the proper tinctures are prepared, appropriate amounts of the prepared tinctures could be mixed into an appropriate amount of delivery matrix in proportions known by those skilled in the art to form a liquid format. The method of use is the delivery by drops or stream of the liquid format to the subject's afflicted eye, generally amounting to 2-5 drops of liquid format a day to each afflicted eye. The invention could be used to treat various symptoms of the eye impairment that occur during aging such as dry eye, redness of the eye, impaired night vision, presbyopia, restricted accommodation, impaired oxygen and nutrient delivery to the crystalline lens, and the like.

The Applicant reports that clinical studies were conducted wherein several subjects suffering from cataracts were given a homeopathic treatment containing Cineraria Maritima. Approximately 2-5 drops of a homeopathic composition containing Cineraria Maritima was placed into a delivery matrix that was subsequently deposited into the afflicted eye daily. After approximately 3 months of treatment, the subjects reported improved vision. Examination of the subjects showed no clinical improvement of the cataract leading to the conclusion that visual improvement could be due to usage of the homeopathic treatment having Cineraria Maritima and may have augmented the eye's power of accommodation. Another conclusion could be reached was that the use of the homeopathic treatment may have caused increased oxygen and possibly nutrient flow to the crystalline lens eye assisting its flexibility.

### CONCLUSION

As noted above, the invention could be used to treat various symptoms of the eye impairment that occur during aging such as dry eye, redness of the eye, impaired night vision, presbyopia, restricted accommodation, impaired oxygen and nutrient delivery to the crystalline lens, and the like.

Although the description above contains many specifications, these should not be construed as limiting the scope of the invention but as merely providing illustrations of some of the presently preferred embodiments of this invention. Thus, the scope of the invention should be determined by the appended claims and their legal equivalents rather than by the examples given.

What is claimed is:

1. A homeopathic composition suitable for the treatment of the symptoms of eye impairment conditions which comprises:
(A) Cineraria Maritima in five times potency;
(B) Calcium Fluoride in thirteen times potency;
(C) Conium Maculatum in six times potency;
(D) Euphrasia Stricta in six times potency;
(E) Gelsemium Sempervirens in six times potency;
(F) Sodium Chloride in six times potency; and
(G) Ruta Gravelens in six times potency.

2. A composition of claim 1 for the treatment of blurry vision.

3. A composition of claim 2 wherein the blurry vision is caused by presbyopia.

4. A composition of claim 2 wherein the blurry vision occurs at night.

5. A composition of claim 1 for the treatment of dry eyes.

6. A composition of claim 1 for the treatment of red eyes.

7. A composition of claim 1 for the improving nutrient and oxygen flow to the crystalline lens.

8. A composition of claim 1 for the treatment of symptoms of presbyopia.

9. A composition of claim 1 for reducing an impairment of the accommodation of the eye.

10. A composition of claim 1 wherein the composition is applied to a person wearing contact lens.

11. A method for symptomatic treatment of eye impairment by the application to the eye of a homeopathic composition comprising:
(A) Cineraria Maritima in five times potency;
(B) Calcium Fluoride in thirteen times potency;
(C) Conium Maculatum in six times potency;
(D) Euphrasia Stricta in six times potency;
(E) Gelsemium Sempervirens in six times potency;
(F) Sodium Chloride in six times potency; and
(G) Ruta Gravelens in six times potency.

12. A method as claimed in claim 11 for treatment of the symptoms of presbyopia.

13. A method as claimed in claim 11 for improving an impaired accommodation of an eye.

14. A method as claimed in claim 11 for symptomatic treatment of dry eyes.

15. A method as claimed in claim 11 for improving the delivery of oxygen and nutrients to a crystalline lens.

16. A method as claimed in claim 11 for symptomatic treatment of red eyes.

17. A method as claimed in claim 11 for improving impaired night vision.

18. A method as claimed in claim 11 for symptomatic treatment of blurry vision.
